(19) United States
(12) Patent Application Publication
(10) Pub. No.: US 2023/0201427 A1
(43) Pub. Date: Jun. 29, 2023

(54) METHOD FOR PREPARING STRUCTURED HYDROGEL AND METHOD FOR PREPARING HYDROGEL HEART VALVE

(71) Applicant: LANZHOU INSTITUTE OF CHEMICAL PHYSICS CAS, Lanzhou (CN)

(72) Inventors: Xiaolong Wang, Lanzhou (CN); Jiayu Wu, Lanzhou (CN); Pan Jiang, Lanzhou (CN); Yaozhong Lu, Lanzhou (CN); Feng Zhou, Lanzhou (CN)

(21) Appl. No.: 18/070,580

(22) Filed: Nov. 29, 2022

(30) Foreign Application Priority Data
Dec. 23, 2021 (CN) 202111591052.7

(51) Int. Cl. A61L 27/52; A61L 27/26; B33Y 10/00; B33Y 70/00; B33Y 40/20; B29C 64/124; B29C 64/30; A61L 27/50

(52) U.S. Cl. CPC A61L 27/52; A61L 27/26; B33Y 10/00; B33Y 70/00; B33Y 40/20; B29C 64/124; B29C 64/30; A61L 27/507; A61L 2430/20; B33Y 80/00

(57) ABSTRACT

The disclosure provides a method for preparing a structured hydrogel and a method for preparing a hydrogel heart valve. In the disclosure, the method includes: providing a photocurable hydrogel ink; establishing a three-dimensional digital model, and conducting photocuring 3D printing on the photocurable hydrogel ink to obtain a printed hydrogel; and immersing the printed hydrogel in water to obtain the structured functional hydrogel, wherein the photocurable hydrogel ink comprises: a high-density hydrogen-bonded unsaturated monomer, a photoinitiator, a dye, and a solvent; and the solvent comprises water and dimethyl sulfoxide.

### CROSS REFERENCE TO RELATED APPLICATION

This patent application claims the benefit and priority of Chinese Patent Application No. 202111591052.7 filed with the China National Intellectual Property Administration on Dec. 23, 2021, the disclosure of which is incorporated by reference herein in its entirety as part of the present application.

### TECHNICAL FIELD

The present disclosure relates to the technical field of hydrogels, and in particularly to a method for preparing a structured hydrogel and a method for preparing a hydrogel heart valve.

### BACKGROUND

In recent years, novel biomaterials such as polymers, ceramics, and metals have achieved rapid development and have been widely used in the medical field, greatly improving the treatment efficiency of many diseases. Although biomaterials are widely used in biomedicine, many biomaterials are still limited in use due to the lack of desirable functional properties such as biomechanical matching, biocompatibility, personalized biofabrication, and surface-interface interactions of biological systems.

Based on the current status and future development of biomaterials, it is necessary to control the design, synthesis, function, and structured fabrication of novel biomaterials, resulting in novel hydrogel-based biomaterials. Hydrogels have a hydrophilic polymer network structure, and water can penetrate between the polymer chains of the hydrophilic polymer network structure, leading to swelling. The advantages of hydrogels in biological applications are due to their high moisture content, biomechanical matching, and biocompatibility. Traditional hydrogels are generally divided into natural hydrogels and synthetic hydrogels. The natural hydrogel comprises polysaccharides, such as cellulose, alginic acid, hyaluronic acid and chitosan, and polypeptides, such as poly-L-lysine, collagen, and poly-L-glutamic acid. The synthetic hydrogel comprises alcohol, acrylic acid and derivatives thereof, such as polyacrylic acid, polymethacrylic acid and polyacrylamide.

The existing hydrogels, whether natural or synthetic, have poor toughness.

### SUMMARY

In view of this, an object of the present disclosure is to provide a method for preparing a structured hydrogel and a method for preparing a hydrogel heart valve. The structured hydrogel prepared by the method according to the present disclosure has excellent toughness.

In order to achieve the above object of the present disclosure, the present disclosure provides the following technical solutions:

The present disclosure provides a method for preparing a structured hydrogel, comprising:
providing a photocurable hydrogel ink;
conducting photocuring 3D printing on the photocurable hydrogel ink according to a predetermined three-dimensional digital model to obtain a printed hydrogel; and
immersing the printed hydrogel in water to obtain the structured hydrogel,
wherein the photocurable hydrogel ink comprises the following components: a monomer, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea, and allyl thiourea; and
the solvent comprises water and dimethyl sulfoxide.

In some embodiments, the monomer further comprises a low-density hydrogen-bonded unsaturated monomer, and the low-density hydrogen-bonded unsaturated monomer comprises acrylamide or acrylic acid.

In some embodiments, a mass ratio of water to dimethyl sulfoxide in the solvent is within a range of 9:1 to 1:9.

In some embodiments, the photoinitiator is a waterborne photoinitiator that comprises at least one selected from the group consisting of a 2959 photoinitiator (2-Hydroxy-4′-(2-hydroxyethoxy)-2-methylpropiophenone), a LAP photoinitiator (lithium phenyl-2,4,6-trimethylbenzoylphosphinate), and a V-50 photoinitiator (2,2′-Azobis(2-methylpropionamidine)dihydrochloride); a mass of the photoinitiator is 0.1-1% of the mass of the monomer.

In some embodiments, a mass percentage content of a solute in the photocurable hydrogel ink is within a range of 5-30%.

In some embodiments, the photocuring 3D printing is performed under parameters comprising: a light source wavelength within a range of 385-405 nm; an exposure time within a range of 5-60 seconds for each layer, and a slice layer thickness within a range of 0.05-0.1 mm

In some embodiments, the immersing the printed hydrogel in water is conducted for 5-15 days.

The present disclosure further provides a method for preparing a hydrogel heart valve, comprising:
providing a photocurable hydrogel ink;
conducting photocuring 3D printing on the photocurable hydrogel ink according to a predetermined three-dimensional digital model of a heart valve to obtain a printed hydrogel;
immersing the printed hydrogel in water to obtain a structured hydrogel; and
mixing the structured hydrogel with a functional monomer, and conducting surface modification to obtain the hydrogel heart valve;
wherein the photocurable hydrogel ink comprises the following components: a monomer, a RAFT reagent, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea, and allyl thiourea;
the solvent comprises water and dimethyl sulfoxide; and
the functional monomer comprises one of sodium p-styrene sulfonate and a heparinoid active monomer.

In some embodiments, the RAFT reagent is a water-soluble RAFT reagent, and the water-soluble RAFT reagent comprises 4-cyano-4-(((ethylthio)carbonothioyl)thio)pentanoic acid, 2-(n-butylthiocarbosulfanylthio)propionic acid, or 4-cyano-4-((dodecylsulfanylthiocarbonyl)sulfanyl)pentanoic acid; and a mass of the RAFT reagent is 0.1-2% of the mass of the monomer.

In some embodiments, the surface modification is conducted at a temperature of 60-90 ° C. for 5 min to 48 h.

The present disclosure provides a method for preparing a structured hydrogel, comprising: providing a photocurable hydrogel ink; conducting photocuring 3D printing on the photocurable hydrogel ink according to a predetermined three-dimensional digital model to obtain a printed hydrogel; and immersing the printed hydrogel in water to obtain the structured hydrogel, wherein the photocurable hydrogel ink comprises the following components: a monomer, a photoinitiator, a dye, and a solvent; the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea, and allyl thiourea; and the solvent comprises water and dimethyl sulfoxide. In the present disclosure, the high-density hydrogen-bonded unsaturated monomer is dissolved in the mixed solvent of dimethyl sulfoxide and water to be configured to the photocurable hydrogel ink. After the photocuring 3D printing, the printed hydrogel is immersed in water, such that dimethyl sulfoxide in the printed hydrogel diffuses into water to conduct phase inversion. Therefore, hydrogen bonds inside the printed hydrogel are reconstructed to improve the toughness of the structured functional hydrogel.

The present disclosure further provides a method for preparing a hydrogel heart valve, comprising: providing a photocurable hydrogel ink; conducting photocuring 3D printing on the photocurable hydrogel ink according to a predetermined three-dimensional digital model of a heart valve to obtain a printed hydrogel; immersing the printed hydrogel in water to obtain a structured hydrogel; and mixing the structured hydrogel with a functional monomer, and conducting surface modification to obtain the hydrogel heart valve, wherein the photocurable hydrogel ink comprises the following components: a monomer, a RAFT reagent, a photoinitiator, a dye, and a solvent; the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea and allyl thiourea; the solvent comprises water and dimethyl sulfoxide; and the functional monomer comprises one of sodium p-styrene sulfonate and a heparinoid active monomer. In the present disclosure, the high-density hydrogen-bonded unsaturated monomer is dissolved in the mixed solvent of the dimethyl sulfoxide and water to be configured to the photocurable hydrogel ink. After the photocuring 3D printing, the printed hydrogel is immersed in water, such that dimethyl sulfoxide in the printed hydrogel diffuses into water to conduct phase inversion. Therefore, hydrogen bonds inside the printed hydrogel are reconstructed to improve the toughness of the structured functional hydrogel. The structured hydrogel is subjected to surface modification with the sodium p-styrene sulfonate or the heparinoid active monomer to improve the cytocompatibility, hemocompatibility, and histocompatibility of the hydrogel heart valve.

### BRIEF DESCRIPTION OF DRAWINGS

FIG. 1 shows a preparation principle diagram of the structured hydrogel provided by the present disclosure.

FIG. 2 shows an optical photograph of the structured hydrogel prepared according to Example 1.

FIG. 3 shows a mechanical property diagram of the structured hydrogel prepared according to Example 1.

FIG. 4 shows a mechanical property diagram of the structured hydrogel prepared according to Example 2.

FIG. 5 shows a mechanical property diagram of the structured hydrogel prepared according to Example 3.

FIG. 6 shows a mechanical property diagram of the structured hydrogel prepared according to Example 3.

FIG. 7 shows an optical photograph of the structured hydrogel prepared according to Example 5.

FIG. 8 shows a mechanical property diagram of the structured hydrogel prepared according to Example 5.

FIG. 9 shows mechanical properties of a structured hydrogel prepared according to Comparative Example 1.

FIG. 10 shows an optical photograph of the hydrogel heart valve prepared according to Example 7.

FIG. 11 shows a mechanical property diagram of the hydrogel heart valve prepared according to Example 7.

FIG. 12 shows an optical photograph of the tubular hydrogel heart valve prepared according to Example 8.

FIG. 13 shows an optical photograph of the heart prepared according to Example 8.

FIG. 14 shows a mechanical property diagram of the hydrogel heart valve prepared according to Example 9.

FIG. 15 shows a mechanical property diagram of the hydrogel heart valve prepared according to Example 10.

FIG. 16 shows an optical photograph of the hydrogel heart valve prepared according to Example 11.

FIG. 17 shows a mechanical property diagram of the hydrogel heart valve prepared according to Comparative Example 4.

FIG. 18 shows a mechanical property diagram of the hydrogel heart valve prepared according to Comparative Example 5.

### DETAILED DESCRIPTION OF THE EMBODIMENTS

The present disclosure provides a method for preparing a structured functional hydrogel, comprising:
providing a photocurable hydrogel ink;
conducting photocuring 3D printing on the photocurable hydrogel ink according to a predetermined three-dimensional digital model to obtain a printed hydrogel; and
immersing the printed hydrogel in water to obtain the structured functional hydrogel,
wherein the photocurable hydrogel ink comprises the following components: a monomer, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide (C₄H₇N₃O₂, NASC), N-acryloyl glycinamide (C₅H₈N₂O₂, NAGA), allyl urea, and allyl thiourea; and
the solvent comprises water and dimethyl sulfoxide.

In some embodiments, in the present disclosure, unless otherwise specified, the raw materials used herein are all commercially available products.

In the present disclosure, a photocurable hydrogel ink is provided.

In the present disclosure, the photocurable hydrogel ink comprises the following components: a monomer, a photoinitiator, a dye, and a solvent.

In the present disclosure, the monomer comprises a high-density hydrogen-bonded unsaturated monomer, and the high-density hydrogen-bonded unsaturated monomer comprises at least one selected from the group consisting of N-acryloyl semicarbazide (C₄H₇N₃O₂, NASC), N-acryloyl glycinamide (C₅H₈N₂O₂, NAGA), allyl urea, and allyl thiourea, preferably N-acryloyl semicarbazide and N-acryloyl glycinamide, more preferably N-acryloyl semicarbazide.

In some embodiments, in the present disclosure, the monomer further comprises a low-density hydrogen-bonded unsaturated monomer, and the low-density hydrogen-bonded unsaturated monomer comprises acrylamide or acrylic acid. In some embodiments of the present disclosure, a mass ratio of the low-density hydrogen-bonded unsaturated monomer to the high-density hydrogen-bonded unsaturated monomer is within a range of (1-5):10, preferably (2-4):10.

In some embodiments, in the present disclosure, the photoinitiator is a waterborne photoinitiator, and the waterborne photoinitiator comprises at least one selected from the group consisting of a 2959 photoinitiator, a LAP photoinitiator, and a V-50 photoinitiator. In some embodiments of the present disclosure, a mass of the photoinitiator is 0.1-1% of the mass of the monomer, preferably 0.5%.

In some embodiments, in the present disclosure, the dye is an aqueous dye that comprises tartrazine or anthocyanin. In some embodiments, in the present disclosure, a mass of the dye is 0.02-0.5% of the mass of the monomer.

In the present disclosure, the solvent comprises water and dimethyl sulfoxide. In some embodiments of the present disclosure, a mass ratio of water to dimethyl sulfoxide in the solvent is within a range of 9:1 to 1:9, preferably 7:3.

In some embodiments, in the present disclosure, a mass percentage content of the solute in the photocurable hydrogel ink is within a range of 5-30%; and the solute refers to all components except a solvent in the photocurable hydrogel ink.

In the present disclosure, after the photocurable hydrogel ink is provided, photocuring 3D printing is conducted on the photocurable hydrogel ink according to a predetermined three-dimensional digital model to obtain a printed hydrogel.

In some embodiments of the present disclosure, the photocuring 3D printing is conducted under parameters comprising: a light source wavelength within a range of preferably 385-405 nm, more preferably 405 nm; an exposure time within a range of preferably 5-60 seconds for each layer, more preferably 20-30 seconds; a slice layer thickness within a range of preferably 0.05-0.1 mm; in some embodiments, the printing is conducted at room temperature, that is, without additional cooling or additional heating.

In the present disclosure, after the printed hydrogel is obtained, the printed hydrogel is immersed in water to obtain the structured hydrogel.

In some embodiments of the present disclosure, the water immersion (i.e. immersing the printed hydrogel in water) is conducted for 5-15 days, preferably 10 days. In some embodiments, the present disclosure, the water immersion is conducted at room temperature, that is, without additional cooling or additional heating. In the present disclosure, the water immersion refers to immersing the printed hydrogel in water to conduct phase inversion.

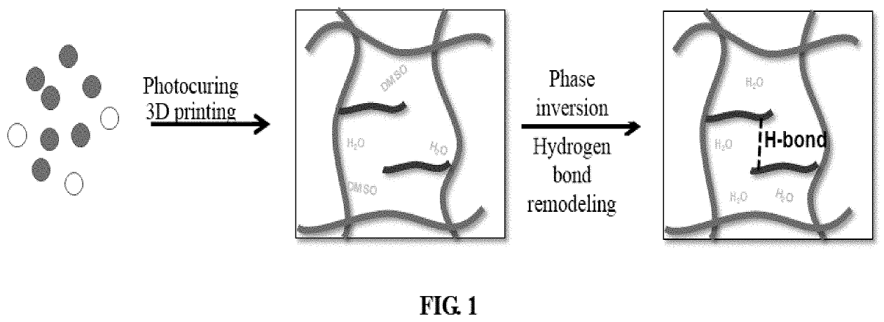

FIG. 1 shows a preparation principle diagram of the structured functional hydrogel provided by the present disclosure.

The present disclosure further provides a method for preparing a hydrogel heart valve, comprising:
providing a photocurable hydrogel ink;
conducting photocuring 3D printing on the photocurable hydrogel ink according to a predetermined three-dimensional digital model of a heart valve to obtain a printed hydrogel;
immersing the printed hydrogel in water to obtain a structured hydrogel; and
mixing the structured hydrogel with a functional monomer, and conducting surface modification to obtain the hydrogel heart valve,
wherein the photocurable hydrogel ink comprises the following components: a monomer, a RAFT reagent, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide (C₄H₇N₃O₂, NASC), N-acryloyl glycinamide (C₅H₈N₂O₂, NAGA), allyl urea, and allyl thiourea;
the solvent comprises water and dimethyl sulfoxide; and
the functional monomer comprises one of sodium p-styrene sulfonate and a heparinoid active monomer.

In the present disclosure, a photocurable hydrogel ink is provided.

In the present disclosure, the photocurable hydrogel ink comprises the following components: a monomer, a RAFT reagent, a photoinitiator, a dye, and a solvent.

In the present disclosure, the monomer comprises a high-density hydrogen-bonded unsaturated monomer, and the high-density hydrogen-bonded unsaturated monomer comprises at least one selected from the group consisting of N-acryloyl semicarbazide (C₄H₇N₃O₂, NASC), N-acryloyl glycinamide (C₅H₈N₂O₂, NAGA), allyl urea, and allyl thiourea, preferably N-acryloyl semicarbazide and N-acryloyl glycinamide, more preferably N-acryloyl semicarbazide.

In some embodiments, in the present disclosure, the monomer further comprises a low-density hydrogen-bonded unsaturated monomer, and the low-density hydrogen-bonded unsaturated monomer comprises acrylamide or acrylic acid. In some embodiments, a mass ratio of the low-density hydrogen-bonded unsaturated monomer to the high-density hydrogen-bonded unsaturated monomer is within a range of (1-5):10, preferably (2-4):10.

In some embodiments, in the present disclosure, the RAFT reagent is a water-soluble RAFT reagent, and the water-soluble RAFT reagent comprises 4-cyano-4- (((ethylthio)carbonothioyl)thio)pentanoic acid, 2-(n-butylthiocarbosulfanylthio)propionic acid, or 4-cyano-4-((dodecylsulfanylthiocarbonyl)sulfanyl)pentanoic acid. In some embodiments, in the present disclosure, a mass of the RAFT reagent is 0.1-2% of the mass of the monomer.

In some embodiments, in the present disclosure, the photoinitiator is a waterborne photoinitiator, and the waterborne photoinitiator comprises at least one selected from the group consisting of a 2959 photoinitiator, a LAP photoinitiator, and a V-50 photoinitiator. In some embodiments of the present disclosure, a mass of the photoinitiator is 0.1-1% of the mass of the monomer, preferably 0.5%.

In some embodiments, in the present disclosure, the dye is an aqueous dye, and the aqueous dye comprises tartrazine, eosin or anthocyanin. In some embodiments, in the present disclosure, a mass of the dye is 0.02-0.5% of the mass of the monomer.

In the present disclosure, the solvent comprises water and dimethyl sulfoxide. In some embodiments of the present disclosure, a mass ratio of water to dimethyl sulfoxide in the solvent is within a range of 9:1 to 1:9, preferably 7:3.

In some embodiments, in the present disclosure, a mass percentage content of the solute in the photocurable hydrogel ink is within a range of 5-30%; the solute refers to all components except a solvent in the photocurable hydrogel ink.

In the present disclosure, after the photocurable hydrogel ink is provided, photocuring 3D printing is conducted on the photocurable hydrogel ink according to a predetermined three-dimensional digital model of a heart valve to obtain a printed hydrogel.

In the present disclosure, the photocuring 3D printing is conducted under parameters comprising: a light source wavelength within a range of preferably 385-405 nm, more preferably 405 nm; an exposure time within a range of preferably 5-60 seconds, more preferably 20-30 seconds for each layer; a slice layer thickness within a range of preferably 0.05-0.1 mm; in some embodiments, the printing is conducted at room temperature, that is, without additional cooling or additional heating.

In the present disclosure, after the printed hydrogel is obtained, the printed hydrogel is immersed in water to obtain a structured hydrogel.

In some embodiments, in the present disclosure, the water immersion is conducted for 5-15 days. In some embodiments, the present disclosure, the water immersion is conducted at room temperature, that is, without additional cooling or additional heating. In the present disclosure, the water immersion refers to immersing the printed hydrogel in water to conduct phase inversion.

In the present disclosure, after the structured hydrogel is obtained, the structured hydrogel is mixed with a functional monomer, and surface modification is conducted to obtain the hydrogel heart valve; wherein the functional monomer comprises one of sodium p-styrene sulfonate and a heparinoid active monomer.

In the present disclosure, the functional monomer comprises one of sodium p-styrene sulfonate and a heparinoid active monomer.

In some embodiments, in the present disclosure, the functional monomer is used in the form of a functional monomer solution; a solvent of the functional monomer solution is a polar solvent, and the polar solvent comprises water, N,N-dimethylformamide, N,N-dimethylacetamide and tetrahydrofuran; and the functional monomer solution has a mass concentration of 5-80%.

In some embodiments, in the present disclosure, the surface modification is conducted at a temperature within a range of 60-90° C., preferably 70-80° C.; in some embodiments, the surface modification is conducted for 5 min to 48 h.

In some embodiments, in the present disclosure, after the surface modification, the method further comprises placing an obtained surface-modified system in water and balancing to obtain the hydrogel heart valve.

In some embodiments, in the present disclosure, the balancing is conducted at room temperature, that is, without additional heating or additional cooling. In some embodiments, in the present disclosure, the balancing is conducted for 12-72 h.

The method for preparing a structured hydrogel and the method for preparing a hydrogel heart valve as provided by the present disclosure will be described in detail below with reference to the examples, but they should not be construed as limiting the protection scope of the present disclosure.

### EXAMPLE 1

15.000 g of N-acryloyl semicarbazide was added into 35.000 g of a mixed solvent of dimethyl sulfoxide and deionized water (with a mass ratio of dimethyl sulfoxide to deionized water being 7:3). After the monomer was completely dissolved, 0.075 g of a photoinitiator (LAP, with a mass of 0.5% of that of the monomer) was added, and 0.010 g of tartrazine was added thereto, obtaining a uniform and transparent photocurable hydrogel ink.

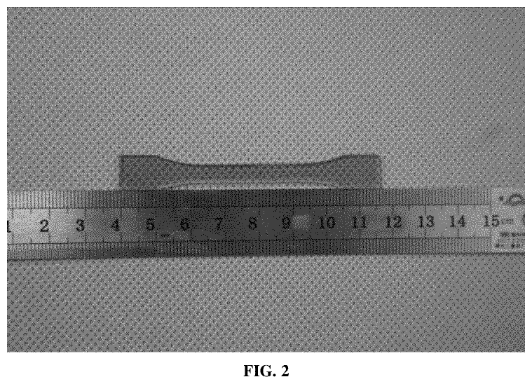

The photocurable hydrogel ink was transferred to the magazine of a photocuring 3D printer. The photocuring 3D printer had a light source wavelength of 405 nm, an exposure time of 20-30 seconds for each layer and a slice layer thickness of 0.1 mm The printing environment was room temperature. A model built by 3D modeling software was imported into a 3D printing software to drive the printer for manufacturing. The resulting printed hydrogel was immersed in deionized water for 10 days, obtaining a structured hydrogel. The optical photograph of the obtained structured hydrogel is shown in FIG. 2.

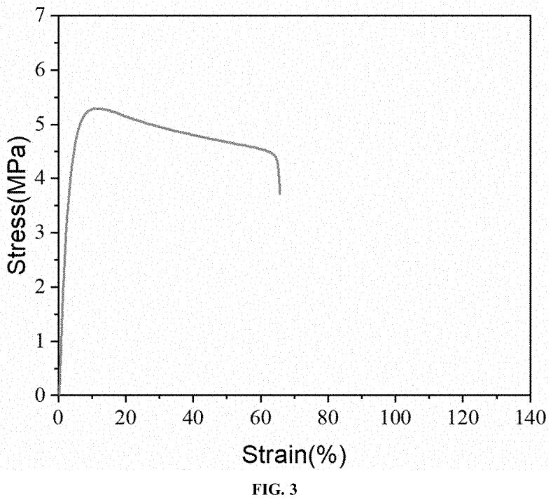

The mechanical properties of the structured hydrogel were tested by a universal material testing machine. The test results are shown in FIG. 3. It can be seen from FIG. 3 that when the strain is 70±53%, the structured hydrogel has a tensile strength of 4.43±0.74 MPa, an elastic modulus of 62.61±12.87 MPa calculated from a stress-strain curve, and a tearing energy of 21.35±0.24 kJ/m².

### EXAMPLE 2

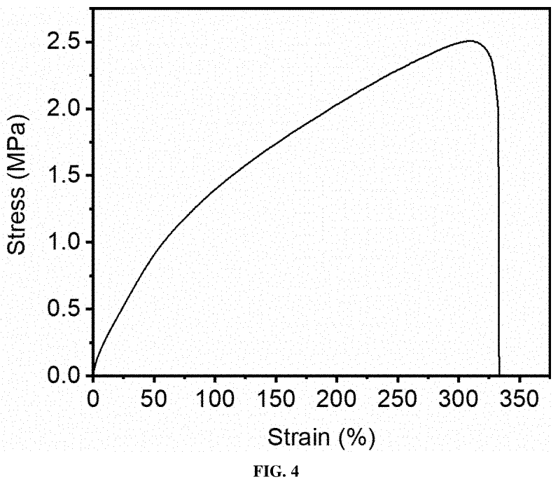

This example was conducted according to Example 1 except that 10.000 g of N-acryloyl semicarbazide and 5.000 g of acrylamide were added. The mechanical properties of the structured hydrogel were tested by a universal material testing machine. The test results are shown in FIG. 4. It can be seen from FIG. 4 that when the strain is 335±63%, the structured hydrogel has a tensile strength of 2.51±0.32 MPa, an elastic modulus of 2.90±0.14 MPa calculated from a stress-strain curve, and a tearing energy of 17.25±0.37 kJ/m².

### EXAMPLE 3

8.330 g of N-acryloyl semicarbazide and 4.170 g of acrylamide were added into 37.500 g of a mixed solvent of dimethyl sulfoxide and deionized water (with a mass ratio of dimethyl sulfoxide to deionized water being 7:3). After the monomer was completely dissolved, 0.063 g of a photoinitiator (LAP, with a mass of 0.5% of that of the monomer) was added, and 0.01 g of tartrazine was added thereto, obtaining a uniform and transparent photocurable hydrogel ink.

Printing and post-treatment were the same as those in Example 1.

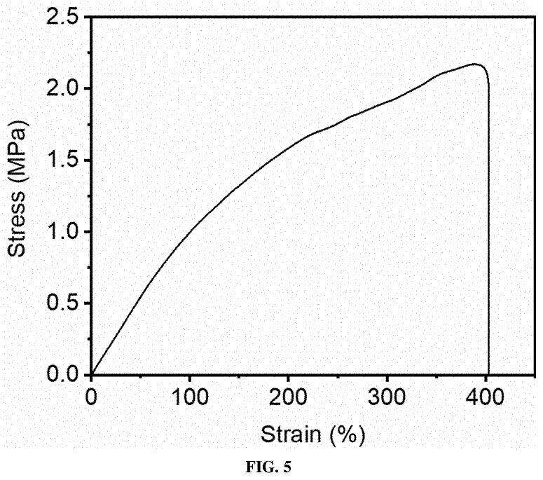

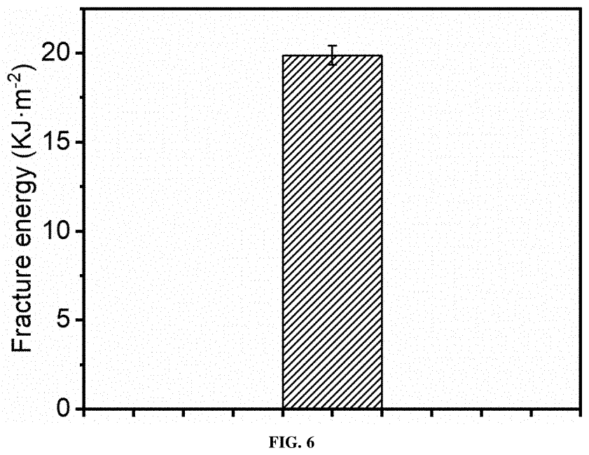

The mechanical properties of the structured hydrogel were tested by a universal material testing machine. The test results are shown in FIG. 5 and FIG. 6. It can be seen from FIG. 5 that when the strain is 410±34%, the structured hydrogel has a tensile strength of 2.06±0.20 MPa, and an elastic modulus of 1.06±0.13 MPa calculated from a stress-strain curve. It can be seen from FIG. 6 that the structured hydrogel has a tearing energy of 19.55±0.51 kJ/m².

### EXAMPLE 4

10.000 g of N-acryloyl semicarbazide and 2.500 g of acrylamide were added into 37.500 g of a mixed solvent of dimethyl sulfoxide and deionized water (with a mass ratio of dimethyl sulfoxide to deionized water being 7:3). After the monomer was completely dissolved, 0.063 g of a photoinitiator (LAP, with a mass of 0.5% of that of the monomer) was added, and 0.010 g of tartrazine was added thereto, obtaining a uniform and transparent photocurable hydrogel ink.

Printing and post-treatment were the same as those in Example 1.

The mechanical properties of the structured hydrogel were tested by a universal material testing machine. When the strain is 557±31%, the structured hydrogel has a tensile strength of 3.25±0.37 MPa, an elastic modulus of 1.93±0.22 MPa calculated from a stress-strain curve, and a tearing energy of 26.35±0.27 kJ/m².

### Example 5

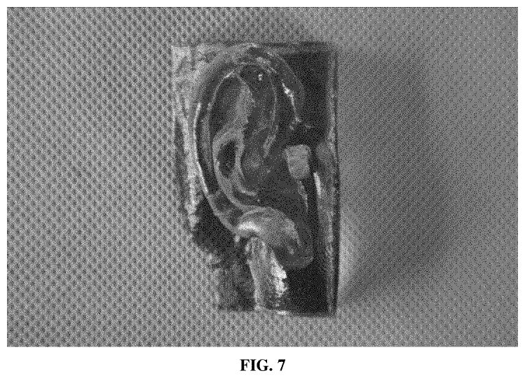

9.615 g of N-acryloyl semicarbazide and 2.885 g of acrylic acid were added into 37.500 g of a mixed solvent of dimethyl sulfoxide and deionized water (with a mass ratio of dimethyl sulfoxide to deionized water being 7:3). After the monomer was completely dissolved, 0.063 g of a photoinitiator (LAP, with a mass of 0.5% of that of the monomer) was added, and 0.010 g of tartrazine was added thereto, obtaining a uniform and transparent photocurable hydrogel ink. An optical photograph of the structured functional hydrogel is shown in FIG. 7.

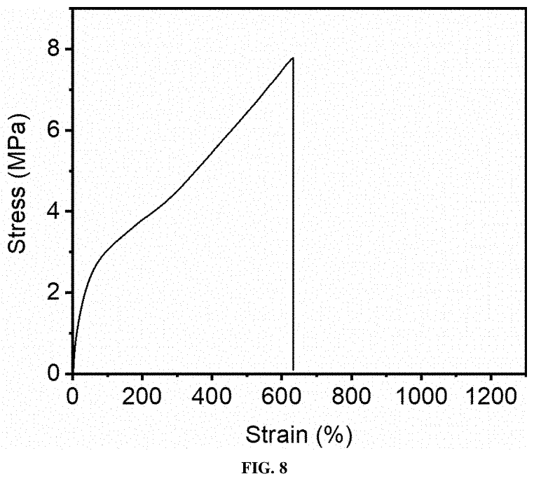

The mechanical properties of the structured hydrogel were tested by a universal material testing machine. The test results are shown in FIG. 8. It can be seen from FIG. 8 that when the strain is 572±55%, the structured hydrogel has a tensile strength of 7.24±0.47 MPa, an elastic modulus of 0.92 MPa calculated from a stress-strain curve, and a tearing energy of 171.10±34 kJ/m².

### EXAMPLE 6

This example was conducted according to Example 5 except that 9.615 g of N-acrylyl carbamide and 2.885 g of N-acryloyl glycinamide were added.

Printing and post-treatment were the same as those in Example 1.

The mechanical properties of the structured hydrogel were tested by a universal material testing machine. When the strain is 407±28%, the structured hydrogel has a tensile strength of 1.82±0.23 MPa, an elastic modulus of 0.65 MPa calculated from a stress-strain curve, and a tearing energy of 18.45±0.46 kJ/m².

### COMPARATIVE EXAMPLE 1

This example was conducted according to Example 1 except that N-acryloyl semicarbazide in Example 1 was replaced by acrylamide.

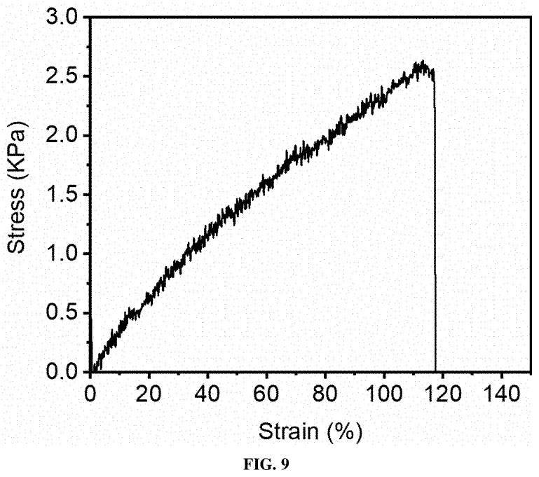

The mechanical properties of the structured hydrogel were tested by a universal material testing machine. The test results are shown in FIG. 9. It can be seen from FIG. 9 that when the strain is 124±31%, the structured hydrogel has a tensile strength of 2.51±0.02 kPa, an elastic modulus of 3.87±1.5 kPa calculated from a stress-strain curve, and a tearing energy of 0.45±0.03 kJ/m².

### COMPARATIVE EXAMPLE 2

This example was conducted according to Example 1 except that dimethyl sulfoxide was omitted.

The mechanical properties of the structured hydrogel were tested by a universal material testing machine. When the strain is 212±34%, the structured hydrogel has a tensile strength of 0.77±0.11 MPa, an elastic modulus of 0.34±0.02 MPa calculated from a stress-strain curves and a tearing energy of 3.27±0.26 kJ/m².

### COMPARATIVE EXAMPLE 3

This example was conducted according to Example 1 except that the water immersion was not conducted.

The mechanical properties of the printed hydrogel were tested by a universal material testing machine. When the strain is 277±28%, the structured hydrogel has a tensile strength of 0.67±0.08 MPa, an elastic modulus of 0.39±0.21 MPa calculated from a stress-strain curve, and a tearing energy of 2.77±0.31 kJ/m².

### EXAMPLE 7

8.3300 g of N-acryloyl semicarbazide and 4.1700 g of acrylamide were added into 37.5000 g of a mixed solvent of dimethyl sulfoxide and deionized water (with a mass ratio of dimethyl sulfoxide to deionized water being 7:3). After the monomer was completely dissolved, 0.0625 g of a photoinitiator (LAP, with a mass of 0.5% of that of the monomer) and 0.0125 g of a RAFT reagent (4-cyano-4-(((ethylthio)carbonothioyl)thio)pentanoic acid, with a mass of 0.1% of that of the monomer) were added, and 0.0100 g of tartrazine was added thereto, obtaining a uniform and transparent photocurable hydrogel ink.

The photocurable hydrogel ink was transferred to the magazine of a photocuring 3D printer. The photocuring 3D printer had a light source wavelength of 405 nm, an exposure time of 20-30 seconds for each layer, and a slice layer thickness of 0.1 mm The printing environment was room temperature. A heart valve model built by 3D modeling software was imported into a 3D printing software to drive the printer for manufacturing. The resulting 3D-printed valve-structured hydrogel was immersed in deionized water for 10 days, obtaining a structured hydrogel heart valve.

The structured hydrogel heart valve was immersed in a sodium p-styrene sulfonate monomer solution (10 g of sodium p-styrene sulfonate in 90 mL of N,N-dimethylformamide as a solvent) for surface modification, and then reacted at 60° C. for 1 h under nitrogen protection.

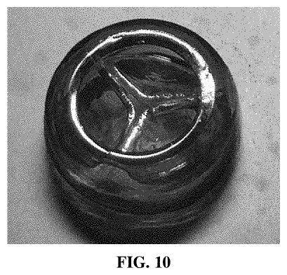

The resulting surface-functionalized hydrogel heart valve was immersed in deionized water for 24 h, and the N,N-dimethylformamide solvent was removed, obtaining a hydrogel heart valve. The specific structure is shown in FIG. 10.

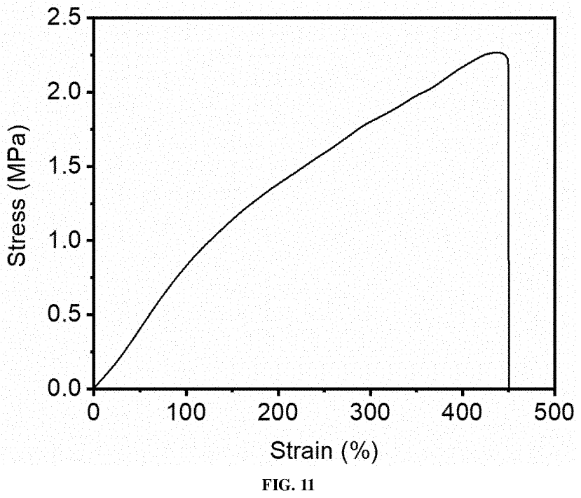

The mechanical properties of the structured hydrogel heart valve were tested by a universal material testing machine. The test results are shown in FIG. 11. It can be seen from FIG. 11 that when the strain is 475±82%, the structured hydrogel heart valve has a tensile strength of 1.83±0.17 MPa, an elastic modulus of 0.64±0.09 MPa calculated from a stress-strain curve, and a tearing energy of 15.77±0.31 kJ/m².

### EXAMPLE 8

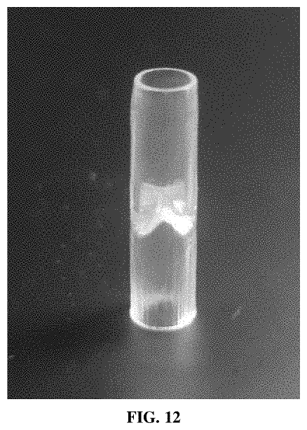

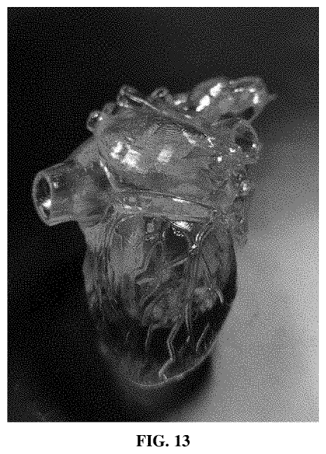

This example was conducted according to Example 7 except that: a tubular hydrogel heart valve and a heart were obtained. The actual pictures are shown in FIG. 12 and FIG. 13.

### EXAMPLE 9

9.3750 g of N-acryloyl semicarbazide and 3.125 g of acrylamide were added into 37.5000 g of a mixed solvent of dimethyl sulfoxide and deionized water (with a mass ratio of dimethyl sulfoxide to deionized water being 7:3). After the monomer was completely dissolved, 0.0625 g of a photoinitiator (LAP, with a mass of 0.5% of that of the monomer) and 0.0125 g of a RAFT reagent (4-cyano-4-(((ethylthio)carbonothioyl)thio)pentanoic acid, with a mass of 0.1% of that of the monomer) were added, and 0.0100 g of tartrazine was added thereto, obtaining a uniform and transparent photocurable hydrogel ink.

Printing and post-treatment were the same as those in Example 7.

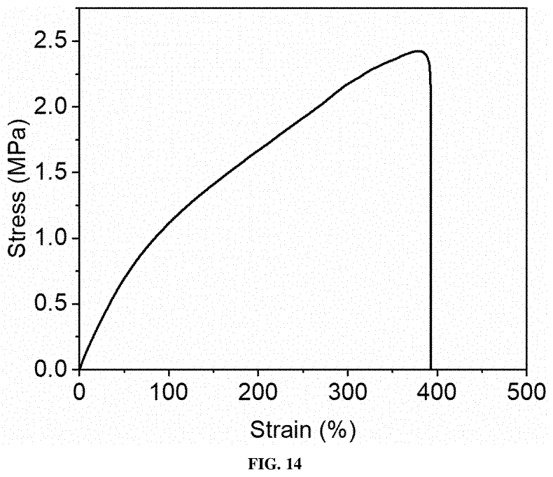

The mechanical properties of the hydrogel heart valve were tested by a universal material testing machine. The test results are shown in FIG. 14. It can be seen from FIG. 14 that when the strain is 405±34%, the structured hydrogel heart valve had a tensile strength of 2.42±0.82 MPa, an elastic modulus of 1.12±0.23 MPa calculated from a stress-strain curve, and a tearing energy of 23.04±0.21 kJ/m².

### EXAMPLE 10

This example was conducted according to Example 7 except that acrylamide was not added.

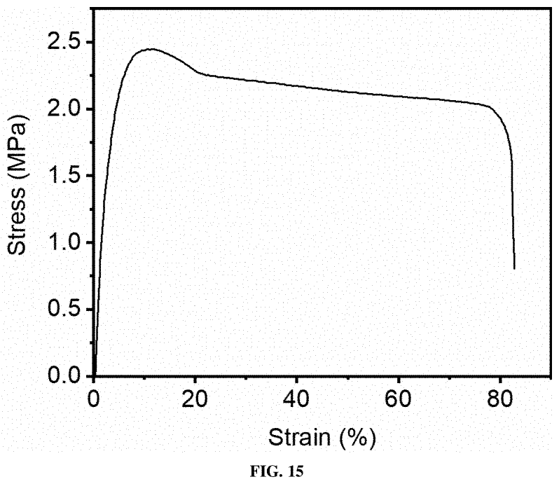

The mechanical properties of the hydrogel heart valve were tested by a universal material testing machine. The test results are shown in FIG. 15. It can be seen from FIG. 15 that when the strain is 83±25%, the structured hydrogel heart valve has a tensile strength of 2.50±0.28 MPa, and an elastic modulus of 33.40±5.79 MPa calculated from a stress-strain curve. The hydrogel heart valve has a tearing energy of 15.28±0.17 kJ/m².

### EXAMPLE 11

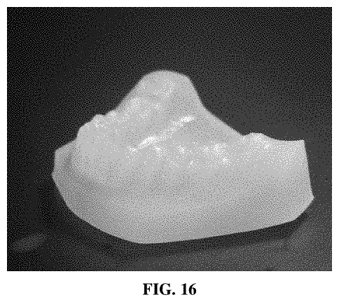

This example was conducted according to Example 7 except that N-acryloyl semicarbazide was replaced by N-acryloyl glycinamide. The actual picture is shown in FIG. 16.

The mechanical properties of the hydrogel heart valve were tested by a universal material testing machine. When the strain is 319±12%, the structured hydrogel heart valve has a tensile strength of 0.55±0.06 MPa, an elastic modulus of 0.13±0.09 MPa calculated from a stress-strain curve, and a tearing energy of 3.55±0.43 kJ/m².

### COMPARATIVE EXAMPLE 4

6.2500 g of N-acryloyl semicarbazide and 6.2500 g of acrylamide were added into 37.5000 g of a mixed solvent of dimethyl sulfoxide and deionized water (with a mass ratio of dimethyl sulfoxide to deionized water being 7:3). After the monomer was completely dissolved, 0.0625 g of a photoinitiator (LAP, with a mass of 0.5% of that of the monomer) and 0.0125 g of a RAFT reagent (4-cyano-4-(((ethylthio)carbonothioyl)thio)pentanoic acid, with a mass of 0.1% of that of the monomer) were added, and 0.0100 g of tartrazine was added thereto, obtaining a uniform and transparent photocurable hydrogel ink.

Printing and post-treatment were the same as those in Example 7.

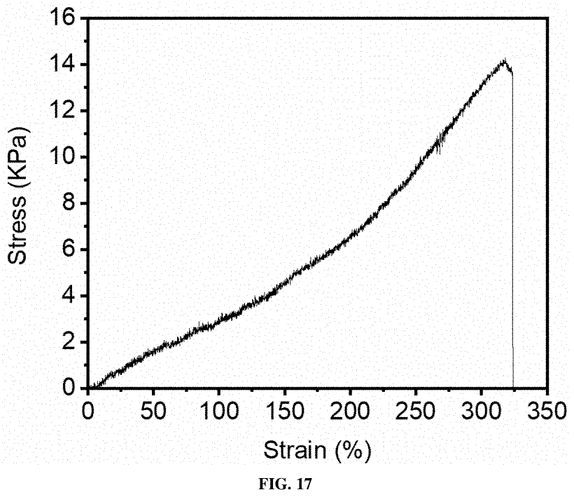

The mechanical properties of the hydrogel heart valve were tested by a universal material testing machine. The test results are shown in FIG. 17. It can be seen from FIG. 17 that when the strain is 325±73%, the structured hydrogel heart valve has a tensile strength of 14.62±1.77 kPa, and an elastic modulus of 6.11±2.15 kPa calculated from a stress-strain curve. The hydrogel heart valve has a tearing energy of 0.77±0.01 kJ/m².

### COMPARATIVE EXAMPLE 5

This example was conducted according to Example 7 except that surface modification was not conducted.

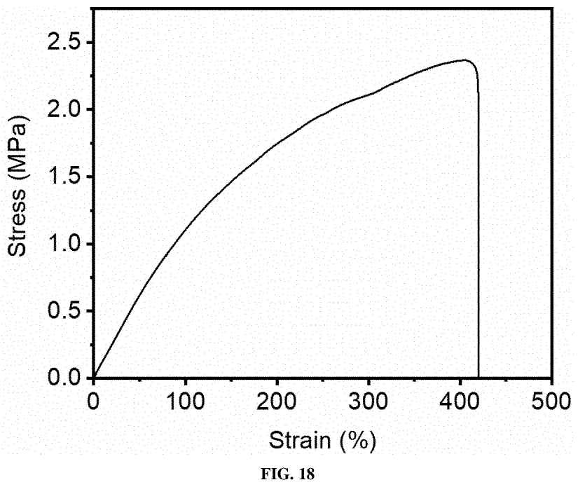

The mechanical properties of the hydrogel heart valve were tested by a universal material testing machine. The test results are shown in FIG. 18. It can be seen from FIG. 18 that when the strain is 442±45%, the structured hydrogel heart valve has a tensile strength of 2.26±0.10 MPa, an elastic modulus of 1.44±0.17 MPa calculated from a stress-strain curve, and a tearing energy of 20.87±0.66 kJ/m².

The above are merely the preferred embodiments of the present disclosure. It should be understood that for those of ordinary skill in the art, several improvements and modifications could be made without departing from the principle of the present disclosure, and those improvements and modifications also should be regarded as falling within the protection scope of the present disclosure.

What is claimed is:

1. A method for preparing a structured hydrogel, comprising:
providing a photocurable hydrogel ink;
conducting photocuring 3D printing on the photocurable hydrogel ink according to a predetermined three-dimensional digital model to obtain a printed hydrogel; and
immersing the printed hydrogel in water to obtain the structured hydrogel,
wherein the photocurable hydrogel ink comprises the following components: a monomer, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea, and allyl thiourea; and
the solvent comprises water and dimethyl sulfoxide.

2. The method according to claim 1, wherein the monomer further comprises a low-density hydrogen-bonded unsaturated monomer, and the low-density hydrogen-bonded unsaturated monomer comprises one of acrylamide and acrylic acid.

3. The method according to claim 1, wherein a mass ratio of water to dimethyl sulfoxide in the solvent is within a range of 9:1 to 1:9.

4. The method according to claim 1, wherein the photoinitiator is a waterborne photoinitiator, and the waterborne photoinitiator comprises at least one selected from the group consisting of a 2959 photoinitiator, a LAP photoinitiator, and a V-50 photoinitiator; and
a mass of the photoinitiator is 0.1-1% of the mass of the monomer.

5. The method according to claim 1, wherein a mass percentage content of a solute in the photocurable hydrogel ink is within a range of 5-30%.

6. The method according to claim 1, wherein the photocuring 3D printing is performed under parameters comprising: a light source wavelength of 385-405 nm, an exposure time of 5-60 seconds for each layer, and a slice layer thickness of 0.05-0.1 mm.

7. The method according to claim 1, wherein immersing the printed hydrogel in water is conducted for 5-15 days.

8. A method for preparing a hydrogel heart valve, comprising:
providing a photocurable hydrogel ink;
conducting photocuring 3D printing on the photocurable hydrogel ink according to a predetermined three-dimensional digital model of a heart valve to obtain a printed hydrogel;
immersing the printed hydrogel in water to obtain the structured hydrogel; and
mixing the structured hydrogel with a functional monomer, and conducting surface modification to obtain the hydrogel heart valve,
wherein the photocurable hydrogel ink comprises the following components: a monomer, a RAFT reagent, a photoinitiator, a dye, and a solvent;
the monomer comprises a high-density hydrogen-bonded unsaturated monomer, and the high-density hydrogen-bonded unsaturated monomer comprises at least one selected from the group consisting of N-acryloyl semicarbazide, N-acryloyl glycinamide, allyl urea, and allyl thiourea;
the solvent comprises water and dimethyl sulfoxide; and
the functional monomer comprises one of sodium p-styrene sulfonate and a heparinoid active monomer.

9. The method according to claim 8, wherein the RAFT reagent is a water-soluble RAFT reagent, and the water-soluble RAFT reagent comprises 4-cyano (((ethylthio)carbonothioyl)thio)pentanoic acid, 2-(n-butylthiocarbosulfanylthio)propionic acid, and 4-cyano-4-((dodecylsulfanylthiocarbonyl)sulfanyl)pentanoic acid; and
a mass of the RAFT reagent is 0.1-2% of the mass of the monomer.

10. The method according to claim 8, wherein the surface modification is conducted at a temperature of 60-90° C.; and the surface modification is conducted for 5 min to 48 h.

11. The method according to claim 2, wherein wherein a mass percentage content of a solute in the photocurable hydrogel ink is within a range of 5-30%.

12. The method according to claim 3, wherein a mass percentage content of a solute in the photocurable hydrogel ink is within a range of 5-30%.

13. The method according to claim 4, wherein a mass percentage content of a solute in the photocurable hydrogel ink is within a range of 5-30%.

14. The method according to claim 4, wherein the photocuring 3D printing is performed under parameters comprising: a light source wavelength of 385-405 nm, an exposure time of 5-60 seconds for each layer, and a slice layer thickness of 0.05-0.1 mm.
